(19) United States
(12) Patent Application Publication
(10) Pub. No.: US 2007/0280986 A1
(43) Pub. Date: Dec. 6, 2007

(54) INTRA-OPERATIVE COATING OF IMPLANTS

(76) Inventors: Carlos Gil, Collierville, TN (US); Aneta Samaranska, Memphis, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/421,579

(22) Filed: Jun. 1, 2006

(51) Int. Cl. A61F 2/02; B05D 3/02

(52) U.S. Cl. 424/423; 427/2.24

(57) ABSTRACT

Embodiments described herein provide methods and systems for applying biologically-active coatings to implants. More particularly, embodiments relate to methods for intra-operative coating of implants, such as orthopedic implants, with biologically-active coatings, such as coatings containing osteoinductive and osteoconductive biological components. An embodiment provides a method for applying a biologically-active coating to an implant. Another embodiment provides a method for implanting an implant with a biologically-active coating during an operation.

### FIELD OF THE INVENTION

Embodiments relate to methods and systems for applying biologically-active coatings to implants. More particularly, embodiments relate to methods for intra-operative coating of orthopedic implants with biologically-active coatings, particularly coatings containing osteoinductive and osteoconductive biological components.

### DESCRIPTION OF RELATED ART

Medical implants or prostheses typically function to replace or augment various structures and tissues in the body. Medical implants include, for example, intervertebral disc replacement devices, spinal fixation systems, facet arthroplasty devices, artificial hips, bone screws, bone plates and rods, prosthetic knee replacements, arterial stents, pacemakers, heart valves, artificial hearts, artificial sphincters, and so forth. The effectiveness of medical implants sometimes is highly dependent upon the implants' interactions with surrounding tissues. For example, in the case of orthopedic implants, it may be desirable that tissue attachment from adjacent bony structures occur at the orthopedic implants' surfaces in order to integrate the implants with the rest of the skeletal system. Therefore, various surface treatments for medical implants, and orthopedic implants in particular, have been proposed in order to stimulate the attachment of a wide variety of tissues to the implants and impart other advantageous in vivo properties to the implants.

The description herein of problems and disadvantages of known apparatus, methods, and devices is not intended to limit the embodiments to the exclusion of these known entities. Indeed, embodiments may include one or more of the known apparatus, methods, and devices without suffering from the disadvantages and problems noted herein.

### SUMMARY

There is a need for methods and systems to apply biologically-active coatings to implants. More particularly, there is a need for methods to intra-operatively coat implants with biologically-active coatings. More particularly, there is a need for intra-operative methods to coat orthopedic implants with biologically-active coatings containing osteoinductive and osteoconductive biological components. Embodiments solve some or all of these needs, as well as additional needs.

Therefore, in accordance with an embodiment, there is provided a method for applying a biologically-active coating to an implant. The method may comprise mixing together a polymerizable component and a biological component to form a polymerizable solution; applying the polymerizable solution to the implant; and curing the polymerizable solution. Mixing, applying, and curing may be carried out during the course of an implant operation.

In another embodiment, there is provided a method for implanting an implant with a biologically-active coating during an operation. The method may comprise providing a sterilized polymerizable component and a sterilized biological component; intra-operatively mixing together the polymerizable component and the biological component to form a polymerizable solution; intra-operatively applying the polymerizable solution to the implant; intra-operatively curing the polymerizable solution; and implanting the implant into a patient during the the course of the operation.

These and other features and advantages of the embodiments will be apparent from the description provide herein.

### DETAILED DESCRIPTION OF THE EMBODIMENTS

The following description is intended to convey a thorough understanding of the various embodiments by providing a number of specific embodiments and details involving methods and systems for the intra-operative coating of an implant. It is understood, however, that the embodiments are not limited to these specifically preferred embodiments and details, which are exemplary only. It is further understood that one possessing ordinary skill in the art, in light of known systems and methods, would appreciate the use of the embodiments for their intended purposes and benefits in any number of alternative embodiments.

As used throughout this disclosure, the singular forms “a,” “an,” and “the” include plural reference unless the context clearly dictates otherwise. Thus, for example, a reference to “a biological component” includes a plurality of such biological components, as well as a single biological component, and a reference to “a polymerizable component” is a reference to one or more polymerizable components and equivalents thereof known to those skilled in the art, and so forth.

It is a feature of an embodiment to provide a method for applying a biologically-active coating to an implant. The method may comprise mixing together a polymerizable component and a biological component to form a polymerizable solution; applying the polymerizable solution to the implant; and curing the polymerizable solution. Mixing, applying, and curing may be carried out during the course of an implant operation.

In another embodiment, there is provided a method for implanting an implant with a biologically-active coating during an operation. The method may comprise providing a sterilized polymerizable component and a sterilized biological component; intra-operatively mixing together the polymerizable component and the biological component to form a polymerizable solution; intra-operatively applying the polymerizable solution to the implant; intra-operatively curing the polymerizable solution; and implanting the implant into a patient during the course of the operation.

What is meant by “intra-operatively” is that the intra-operatively performed procedure (e.g., coating of an implant that is to be implanted) takes place during the course of an operation or surgery, or at least close in time to the operation or surgery. Preferably, the “intra-operatively” performed procedure takes place within twenty-four hours of the start of surgery. More preferably, the “intra-operatively” performed procedure takes place within six hours of the start of surgery. Even more preferably, the “intra-operatively” performed procedure takes place within one hour of the start of surgery. Most perferably, the “intra-operatively” performed procedure takes place during the course of surgery. The intra-operative procedure (e.g., coating of the implant) need not take place in the operating room, but instead may occur in another room or facility, as appropriate.

The embodiments are applicable to a wide variety of implants. In particular, the embodiments are applicable to orthopedic implants, for example, articular cups (e.g., acetabular cups), bone pins, bone screws, dental prostheses, elbow implants, facet arthroplasty devices, femoral head implants, femoral stem implants, finger implants, hip screws, hip sockets, humeral head implants, humeral stem implants, intervertebral fusion cages, intramedullary nails, knee minicus implants, pins (e.g., clavicle and hip pins), components of spinal fixation systems (e.g., screws. nails, bone plates etc.), toe implants, total ankle replacement devices, total knee replacement devices, wrist implants, and so forth. One of skill in the art will appreciate many other orthopedic implants that may be used in the embodiments, in accordance with the guidelines herein.

The implants that are useful in the embodiments may be produced from a wide variety of materials, to which coating treatments may be applied. For example, the implants may be fabricated from medical plastics such as polyvinyl chlorides. polypropylenes, polystyrenes, acetal copolymers, polyphenyl sulfones, polycarbonates, acrylics, silicone polymers, polyetheretherketone (PEEK), polyurethanes, polyethylenes, (e.g., ultra high molecular weight polyethylene), polyethylene terphalate (PET), polyethers, polymethylmethacrylate (PMMA), and mixtures and combinations thereof. Medical metals and metal alloys such as titanium, titanium alloys, tantalum, tantalum alloys, stainless steel alloys, cobalt-based alloys, cobalt-chromium alloys, cobalt-chromium-molybdenum alloys, niobium alloys, zirconium alloys, and shape memory alloys such as nitinol also may be used to fabricate the implants. Additionally, ceramics such as alumina, zirconia, hydroxyapatite, and calcium phosphate may be used. Also, natural substrates such as allograft, xenograft, and autograft tissues may be used to fabricate the medical implants. Implants useful in the embodiments also may be composites of medical plastics, metals, alloys, ceramics, and natural tissues, particularly composites comprising carbon fibers or hydroxyapatite polymers. Methods for producing implants are well known in the art and are largely dictated by the particular device, as will be appreciated by one of skill in the art.

Polymerizable components that may be used in the embodiments preferably are biologically-compatible when polymerized so that the implants are appropriate for in vivo use in a human body. Because the polymerizable components preferably are applied to the implant and cured intra-operatively, the polymerizable components preferably also are capable of being cured in a relatively short period of time. For example, in a preferred embodiment, the polymerizable solution formed by mixing one or more polymerizable components with one or more biological components is capable of being cured in less than or equal to about five minutes.

The polymerizable component may be selected from a wide variety of polymerizable materials, in accordance with the embodiments herein. For example, the polymerizable component may be a catalyst-initiated polymerizable component, a redox-initiated polymerizable component, a two-part polymerizable component, a heat-curable polymerizable component, or a radiation-curable polymerizable component. A UV-polymerizable component is a preferred polymerizable component for the use in the embodiments.

One or more polymerizable components may be mixed with one or more biological components to form a polymerizable solution. In the case of a two-part polymerizable component, mixing together the polymerizable component and a biological component may comprise mixing together both parts of the two-part polymerizable polymer with the biological component. Furthermore, in the case of self-polymerizing components (e.g., redox-initiated and two-part polymerizable components) mixing of the polymerizable components with the biological components and application of the polymerizable solution to the implant preferably is carried out relatively quickly in order to apply the solution to the implant before cross-linking (i.e., curing).

After one or more biological components and one or more polymerizable components are mixed to form a polymerizable solution, the solution may be applied to the implant. One of ordinary skill in the art will appreciate the wide variety of methods by which the polymerizable solution may be applied to the implants. For example, the solution may be sprayed or pasted onto the implant, or the implant may be dip-coated in the solution. Alternatively, the polymerizable solution could be molded and cured intraoperatively, based on the implant specifications at the time of surgery, and then the molded and cured material applied to the implant (e.g., by placing it around the implant, or fitting it on the implant).

In a preferred embodiment the polymerizable solution may be applied to the implant by pouring the solution into a mold, press-fitting the implant into the mold, and closing the mold to hold the implant in place. Thus, the coating may be cured while the implant is held in the mold. The size of the mold may be adjusted in order to affect the thickness of the resulting coating (e.g., a larger mold relative to the size of the implant held therein will result in a thicker coating).

If a mold is to be used to apply the polymerizable solution to the implant and a preferred UV-polymerizable component is desired to be used in the polymerizable solution, then the mold preferably is translucent or transparent or made from a UV-penetrable material so that UV-radiation is able to penetrate the mold and cause polymerization of the UV-polymerizable solution therein. Likewise, if another type of radiation-curable polymerizable component is to be used, the mold preferably is penetrable by the required form of radiation. If a mold is to be used to apply the polymerizable solution to the implant and a heat-curable polymerizable component is desired to be used in the polymerizable solution, then the mold preferably is relatively thermally conductive and/or provided with a heating mechanism internal to the mold in order to affect heating of the polymerizable solution. If a mold is to be used to apply the polymerizable solution to the implant and a catalyst-initiated polymerizable component is desired to be used in the polymerizable solution, then the mold preferably is designed with access ports or some other mechanism in order to introduce a catalyst to the polymerizable solution in the mold. Alternatively, the catalyst may be added to the mold just before the mold is closed.

Following application of the polymerizable solution to the implant, the solution may be cured. Because the implant preferably is coated intra-operatively, it also is preferred that the polymerizable solution be capable of curing within a relatively short period of time. For example, it is preferred that the polymerizable solution be sufficiently curried in less than about 1 hour, more preferably less than about one-half an hour, even more preferably less than about one-quarter an hour, and most preferably less than or about five minutes.

One of ordinary skill in the art will recognize that the manner of curing of the polymerizable solution will be dependent upon the type of polymerizable components that are used in the polymerizable solution. For example, in the case of a two-part polymerizable component, curing the polymerizable solution containing the two-part component may comprise allowing the two-part polymerizable polymer in the polymerizable solution to cross-link with itself. Likewise for redox-initiated polymerizable components, curing the polymerizable solution containing the component may comprise allowing the redox-initiated polymerizable components to cross-link with itself. In the case of radiation-curable polymerizable components such as UV-polymerizable components, curing the polymerizable solution containing the components may comprise applying the appropriate form of radiation, for example UV-radiation, to the solution. Curing a polymerizable solution comprising a heat-curable polymerizable component may comprise applying thermal energy to the solution, and curing a polymerizable solution containing a catalyst-initiated polymerizable component may comprise mixing the catalyst with the solution,

In an alternative embodiment, the polymerizable solution may be intra-operatively cured before the cured polymer is placed on the implant. This may be desirable, for example, if the curing process would damage the implant or biological components thereof. In this embodiment, because the polymerizable solution is cured away from the implant, the curing process would not damage the implant. Following curing, the cured polymer may be applied, preferably intra-operatively, to the implant and secured thereto, as will be appreciated by one of ordinary skill in the art.

In a preferred embodiment, the polymerizable components are capable of forming a hydrogel when cured. For example, the polymerizable component may form a polyethylene glycol (PEG) or polyethylene glycol diacrylate (PEGDA) hydrogel upon polymerization. Other suitable hydrogels also may include those hydrogels formed from polyvinyl alcohol; polyacrylamides; polyacrylic acid; poly(acrylonitrile-acrylic acid); polyurethanes; polyethyleneoxide; poly(N-vinyl-2-pyrrolidone); polyacrylates such as poly(2-hydroxy ethyl methacrylate) and copolymers of acrylates with N-vinyl pyrrolidone; N-vinyl lactams; acrylamide; polyurethanes; polyacrylonitrile; other similar materials that form a hydrogel; and mixtures and combinations thereof. Applicable hydrogels also may include xerogel materials, such as those disclosed in U.S. Pat. Nos. 5,047,055, 5,192,326, 5,976,186, 6,264,695, 6,660,827, and 6,726,721, the disclosures of each of which are incorporated by reference herein in their entirety. The hydrogel materials further may be cross-linked to provide additional strength to the coating.

In another preferred embodiment, the polymerizable component preferably may be biodegradable so that, upon implantation of the implant coated with the cured polymer, the polymer eventually will be degraded by the body and removed. A further advantage of a biodegradable polymerizable component is that degradation of the polymer may release the biological component from the coating, thus allowing the biological component to interact with adjacent tissues and increasing the potency of the biological component.

For example, degradable polymers have been described in U.S. Pat. No. 4,716,203 (a PGA-PEG-PGA block copolymer and PGA-PEG diblock copolymers) and U.S. Pat. No. 4,526,938 (non-crosslinked materials with MW in excess of 5,000 based on compositions with PEG), the disclosures of each of which are incorporated herein by reference in their entirety. Other degradable polymers that have been described include terpolymers of d,l-lactide glycolide, and s-caprolactone; PEG copolymerized with lactide, glycolide, and a-caprolactone; and PLA-PEG copolymers. Degradable materials of biological origin such as crosslinked gelatin, hyaluronic acid and derivatives thereof (e.g., as disclosed by U.S. Pat. Nos. 4,987,744 and 4,957,744, the disclosures of each of which are incorporated herein by reference in their entirety), collagen, albumin, keratin, elastin, silk, proteoglycan, glucomannan gel, and polysaccharides such as cross-linked carboxyl-containing polysaccharides also have been described. Furthermore, U.S. Pat. Nos. 5,410,016; 5,529,914; 5,232,984; 5,380,536; 5,573,934; 5,612,050; 5,837,747; 5,846,530; and 5,858,746 generally relate to hydrogels prepared from biodegradable and biostable polymerizable macromers and are incorporated herein by reference in their entirety. Biodegrable polymers such as those described herein may be applicable in the embodiments.

Still other polymerizable materials that may be used in the embodiments may include polyurethanes such as thermoplastic polyurethanes, aliphatic polyurethanes, segmented polyurethanes, hydrophilic polyurethanes, polyether-urethane, polycarbonate-urethane, and silicone polyurethane copolymers; and polyolefins such as polyisobutylene rubber, polyisoprene rubber, neoprene rubber, nitrile rubber, vulcanized rubber, and combinations thereof.

Non-limiting examples of thermoplastic silicone polyurethane copolymers that may be useful in the embodiments include, but are not limited to, polyether silicone polyurethanes; polycarbonate silicone polyurethanes; poly(tetramethylene-oxide) (PTMO) polyether-based aromatic silicone polyurethanes; polydimethylsiloxane (PDMS) polyether-based aromatic silicone polyurethanes; PTMO polyether-based aliphatic silicone polyurethanes; PDMS polyether-based aliphatic silicone polyurethanes; silicone polyurethane ureas; and combinations thereof. Suitable thermoplastic silicone polyurethane copolymers are also commercially available, and non-limiting commercially available, suitable thermoplastic silicone polyurethane copolymers comprise, or alternatively consist of, PURSIL® (including PurSil-10, -20, and -40) (Polymertech, Berkley, Calif.), CARBOSIL® (including CarboSil-10, -20, and -40) (Polymertech, Berkley, Calif.), Elast-Eon silicone polyurethanes with silicone content between 10% and 50% (Aortech Biomaterials, Victoria, Australia), and combinations thereof.

One of ordinary skill in the art will recognize still other polymerizable components that may be used in accordance with the embodiments.

One or more polymerizable components may be mixed with one or more biological components to form a polymerizable solution. These biological components, for example, may facilitate endogenous tissue in-growth and on-growth (i.e., growth of tissue onto and/or into the implant). Preferably, the biological components are osteoinductive or osteoconductive components for promoting bone in-growth and on-growth (i.e., growth of cancellous or cortical bone onto and/or into the implant) or aid in the prevention of bone resorption. Described herein are some exemplary biological components for use in the polymerizable solutions of the embodiments.

Bone morphogenic factors are preferred biological components for use in the polymerizable solutions of the embodiments. Bone morphogenetic factors are growth factors whose activity are specific to bone tissue including, but not limited to, demineralized bone matrix (DBM), bone protein (BP), bone morphogenetic protein (BMP), and mixtures and combinations thereof. Additionally, formulations for promoting the attachment of endogenous bone may comprise bone marrow aspirate, bone marrow concentrate, and mixtures and combinations thereof. Methods of obtaining bone marrow aspirates as well as devices facilitating extraction of bone marrow aspirate are well known in the art and are described, for example, in U.S. Pat. No. 5,257,632, which is incorporated herein by reference in its entirety. Methods for producing DBM also are well known in the art, and DBM may be obtained following the teachings of U.S. Pat. No. 5,073,373, incorporated herein by reference in its entirety, or by obtaining commercially available DBM formulations such as, for example, AlloGro®, commercially available from AlloSource, Centennial, Colo.

BMPs are a class of proteins thought to have osteoinductive or growth-promoting activities on endogenous bone tissue, or function as pro-collagen precursors. Known members of the BMP family that may be utilized as biological components in the polymerizable solutions include, but are not limited to, BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, and BMP-18 polynucleotides and polypeptides, as well as mature polypeptides and polynucleotides encoding the same. The BMPs may be included in the polymerizable solutions as full length BMPs or fragments thereof, or combinations or mixtures thereof, or as polypeptides or polynucleotides encoding the polypeptide fragments of all of the recited BMPs.

Osteoclastogenesis inhibitors inhibit bone resorption by osteoclasts of the bone tissue surrounding the site of implantation, and therefore may be useful as biological components in the polymerizable solutions of the embodiments. Osteoclast and Osteoclastogenesis inhibitors include, but are not limited to, Osteoprotegerin polynucleotides and polypeptides, as well as mature Osteoprotegerin polypeptides and polynucleotides encoding the same. The Osteoprotegerin protein specifically binds to its ligand, osteoprotegerin ligand (TNFSF11/OPGL), both of which are key extracellular regulators of osteoclast development. Osteoclastogenesis inhibitors further include, but are not limited to, chemical compounds such as bisphosphonate, 5-lipoxygenase inhibitors such as those described in U.S. Pat. Nos. 5,534,524 and 6,455,541, heterocyclic compounds such as those described in U.S. Pat. No. 5,658,935, 2,4-dioxoimidazolidine and imidazolidine derivative compounds such as those described in U.S. Pat. Nos. 5,397,796 and 5,554,594, sulfonamide derivatives such as those described in U.S. Pat. No. 6,313,119, and acylguanidine compounds such as those described in U.S. Pat. No. 6,492,356. The preceding patents are incorporated herein by reference in their entirety.

Other growth factors, agents, and compounds may be included in the polymerizable solutions as biological components. Non-limiting examples of such agents that may be included in the polymerizable solutions are hydroxyapatite (HA), tricalcium phosphate (TCP), collagen, platelet derived growth factor (PDGF), transforming growth factor b (TGF-b), insulin-related growth factor-I (IGF-I), insulin-related growth factor-II (IGF-II), fibroblast growth factor (FGF, bFGF, etc.), beta-2-and microglobulin (BDGF II), fibronectin (FN), osteonectin (ON), endothelial cell growth factor (ECGF), cementum attachment extracts (CAE), ketanserin, human growth hormone (HGH), animal growth hormones, epidermal growth factor (EGF), and human alpha thrombin.

Still other examples of biological components that may be added to the polymerizable solution are biocidal/biostatic sugars such as dextran and glucose. peptides; nucleic acid and amino acid sequences such as leptin antagonists, leptin receptor antagonists, and antisense leptin nucleic acids; vitamins; inorganic elements; co-factors for protein synthesis; hormones; endocrine tissue or tissue fragments; synthesizers; enzymes such as collagenase, peptidases, and oxidases; polymer cell scaffolds with parenchymal cells; angiogenic agents; antigenic agents; cytoskeletal agents; cartilage fragments; living cells such as chondrocytes, bone marrow cells, mesenchymal stem cells, natural extracts, genetically engineered living cells, or otherwise modified living cells; autogenous tissues such as blood, serum, soft tissue, and bone marrow; bioadhesives; periodontal ligament chemotactic factor (PDLGF); somatotropin; bone digesters; antitumor agents and chemotherapeutics such as cis-platinum, ifosfamide, methotrexate, and doxorubicin hydrochloride; immuno-suppressants; permeation enhancers such as fatty acid esters including laureate, myristate, and stearate monoesters of polyethylene glycol; bisphosphonates such as alendronate, clodronate, etidronate, ibandronale, (3-amino-1-hydroxypropylidene)-1,1-bisphosphonate (APD), dichloromethylene bisphosphonate, aminobisphosphonatezolendronate, and pamidronate; pain killers and anti-inflammatories such as non-steroidal anti-inflammatory drugs (NSAID) like ketorolac tromethamine, lidocaine hydrochloride, bipivacaine hydrochloride, and ibuprofen: antibiotics and antiretroviral drugs such as tetracycline, vancomycin, cephalosporin, erythromycin, bacitracin, neomycin, penicillin, polymycin B, biomycin, chloromycetin, streptomycin, cefazolin, ampicillin, azactam, tobramycin, clindamycin, gentamicin, and aminoglycocides such as tobramycin and gentamicin; and salts such as strontium salt, fluoride salt, magnesium salt, and sodium salt.

One skilled in the art will appreciate still other advantageous biological components, and in particular other osteoinductive and osteoconductive components, that may be added to the polymerizable solutions.

The embodiments may present a number of different advantages over extra-operative coating of implants. Implants that are coated with biologically active coatings extra-operatively, or well in advance of surgery, often must be stored in a carefully controlled environment in order to preserve the potency of the biological components that are contained in the coating. For example, temperature, humidity/moisture level, oxygen level, light exposure (e.g.. UV) and other variables of the environment in which the extra-operatively coated implants are stored may need to be controlled in order to maintain the biological components' potency. Failure to adequately control these variables may decrease the activity of the biological components in the coatings, and may result in less successful or poor surgical outcomes for patients that receive the degraded extra-operatively coated implants. Furthermore, failure to adequately control these variable may so degrade the implants as to render them unusable, thus necessitating either re-coating of the implants or, if re-coating is not economically or physically feasible, wasteful disposal of the degraded implants.

Even if the environmental variables are adequately regulated, doing so may be prohibitively expensive. Implants that are extra-operatively coated with biologically-active coatings may not be surgically implanted for weeks, months, or an even longer period of time after the coating process is completed. During this time period, the extra-operatively coated implants may be stored by manufacturers, distributors, hospitals, and any other parties through which possession of the implants may pass. Thus, all of these parties must have the capabilities and expertise necessary to provide carefully regulated storage conditions for the implants. Furthermore, different implants, because they may comprise different biological agents, may require different storage conditions. Thus, these parties (e.g., manufacturers, distributors, hospitals, etc.) may need to be able to provide multiple different storage environments for the different types of implants the parties may possess at any given time. Thus, these parties may require extensive investments in expertise and equipment in order to properly handle and store extra-operatively coated medical implants.

Additionally, because extra-operatively coated implants may incorporate biological components that require mutually-exclusive storage conditions, in some cases it may be impossible to combine certain biological components in an implant coating for any extended period of time.

Still another possible shortcoming of extra-operatively coated implants is that the implants, in order to be stored for extended periods of time, often must be sterilized. In particular, it may be difficult to separately sterilize the storage/packaging systems in which the implants are to be kept and then package and seal the implants in the storage/packaging systems without contaminating the implants. Thus, in practice, the storage/packaging system and the implant sealed therein typically must be sterilized following assembly of the packaging system. Sterilization typically is carried out by the application of gamma radiation or gaseous ethylene oxide. Other appropriate sterilizing agents include electron-beam (E-beam) radiation, gas plasma, ultraviolet radiation (UV), and hydrogen peroxide (H₂O₂). However, these sterilizing agents themselves may cause degradation of the biologically-active components or adversely affect the polymerization components used to form the coating, in the extra-operatively applied implant coating. One of ordinary skill in the art may recognize still other shortcomings of extra-operative coating of implants.

The intra-operative coating methods provided in the embodiments may solve or reduce some or all of these problems. Implants that are coated with a biologically-active coating during the course of the implant surgery may be outside of the body in their coated form for only a brief period of time compared to extra-operatively coated implants. Thus, because there is a decreased time period during which degradation of the biological components in the coating may occur, the intra-operatively coated implants may not need to be maintained in the closely regulated environment that extra-operatively coated implants may require. Thus, the intra-operatively coated implants may be more cost-efficient to manufacture and store, and may provide enhanced biological activity (or the same biological activity even with less biological components) compared to extra-operatively coated implants.

Furthermore, the intra-operatively coated implants may not require sterilization subsequent to coating of the implant. This may be accomplished, for example, by sterilizing the polymerizable component, biological components, and implant before mixing the polymer and biological component together, applying the polymerizable solution to the implant, and curing the solution on the implant. Sterilization of the one or more polymerizable components and the one or more biological components may be carried out extra-operatively or intra-operatively. One of ordinary skill in the art will recognize that different biological components require different sterilization procedures, and will recognize what these procedure are. Further, one of ordinary skill in the art will recognize how a polymerizable material and an implant may be sterilized (e.g., the application of gamma radiation, gaseous ethylene oxide, electron-beam (E-beam) radiation, gas plasma, ultraviolet radiation (UV), and hydrogen peroxide (H₂O₂)). Because the polymerizable component, biological component, and implant may be sterilized before mixing, application, and curing occurs, the resulting intra-operatively coated implant may be sufficiently sterile to not need subsequent sterilization before being implanted into a patient. Furthermore, because the intra-operatively coated implant preferably is implanted within a relatively short period of time once the coating has been applied, the implant may not need to be packaged in a sterile manner, and thus may not require sterilization of the assembled packaging system and implant. Thus, the intra-operatively coated implants may be less expensive to store and have a greater biological activity compared to similarly constituted extra-operatively coated implants.

Another benefit of intra-operatively applied coatings may be that the implant may be intra-operatively modified by a surgeon prior to coating. It is not unusual that implants require customization before final implantation in a body. If the implant has been extra-operatively coated, customization may be limited or impossible because doing so may damage the extra-operatively applied coating. Using the methods of the embodiments herein, however, the implant may be modified or customized intra-operatively, (e.g., by cutting away portions of the implant to custom fit the implant), and then the implant also may be coated intra-operatively. Thus, customization may be carried out without damaging the implant's coating, because the implant is not coated until after it has been modified.

Yet another possible advantage of the intra-operatively applied coatings is that they may be applied to any type of implant surface. The polymerizable solution preferably is applied to the implant and cured thereon so that the solution encases or surrounds the implant or a substantial portion thereof. Thus, the cured coating may be held in place on the implant by virtue of its physical structure, rather than by chemical bonding with the implant surface. Thus, chemical interaction between the implant surface and the coating may not be necessary in order to retain the coating on the implant.

One of ordinary skill in the art may recognize still other benefits of intra-operative coating of implants, compared to extra-operative coating of implants.

The foregoing detailed description is provided to describe the embodiments in detail, and is not intended to limit the embodiments. Those skilled in the art will appreciate that various modifications may be made to the embodiments without departing significantly from the spirit and scope thereof.

What is claimed is:

1. A method for applying a biologically-active coating to an implant, comprising:
mixing together a polymerizable component and a biological component to form a polymerizable solution;
applying the polymerizable solution to the implant; and
curing the polymerizable solution;
wherein mixing, applying, and curing are carried out intra-operatively.

2. The method of claim 1, wherein the polymerizable component is selected from the group consisting of catalyst-initiated polymerizable components, redox-initiated polymerizable components, two-part polymerizable components, heat-curable polymerizable components, and radiation-curable polymerizable components.

3. The method of claim 1, wherein applying the polymerizable solution to the implant is selected from the group consisting of spraying the polymerizable solution onto the implant, pasting the polymerizable solution onto the implant, dip-coating the implant in the polymerizable solution, and curing the polymerizable solution and applying the cured polymerized solution to the implant.

4. The method of claim 1, wherein curing the polymerizable solution is selected from the group consisting of applying heat to the polymerizable solution, applying radiation to the polymerizable solution, and introducing a catalyst into the polymerizable solution.

5. The method of claim 1, wherein applying the polymerizable solution to the implant comprises pouring the polymerizable solution into a mold, press-fitting the implant into the mold, and closing the mold.

6. The method of claim 5, wherein the polymerizable solution is cured while the implant is held in the closed mold.

7. The method of claim 1, wherein the polymerizable component is a UV-polymerizable component, and curing the polymerizable solution comprises applying UV-radiation to the polymerizable solution.

8. The method of claim 1, further comprising sterilizing the polymerizable component and the biological component prior to mixing them together.

9. The method of claim 8, wherein sterilizing is carried out extra-operatively.

10. The method of claim 1, wherein the polymerizable solution forms a hydrogel upon curing.

11. The method of claim 10, wherein the hydrogel is selected from polyethylene glycol (PEG) and polyethylene glycol diacrylate (PEGDA).

12. The method of claim 1, wherein the biological component is an osteoinductive or osteoconductive component.

13. The method of claim 1, wherein the polymerizable solution is capable of being cured in less than or about 5 minutes.

14. The method of claim 1, wherein the implant is an orthopedic implant.

15. A method for implanting an implant with a biologically-active coating during an operation, comprising:
providing a sterilized polymerizable component and a sterilized biological component;
intra-operatively mixing together the polymerizable component and the biological component to form a polymerizable solution;
intra-operatively applying the polymerizable solution to the implant;
intra-operatively curing the polymerizable solution; and
implanting the implant into a patient during the course of the operation.

16. The method of claim 15, wherein the polymerizable component is selected from the group consisting of catalyst-initiated polymerizable components, redox-initiated polymerizable components, two-part polymerizable components, heat-curable polymerizable components, and radiation-curable polymerizable components.

17. The method of claim 15, wherein applying the polymerizable solution to the implant is selected from the group consisting of spraying the polymerizable solution onto the implant, pasting the polymerizable solution onto the implant, dip-coating the implant in the polymerizable solution, and curing the polymerizable solution and applying the cured polymerized solution to the implant.

18. The method of claim 15, wherein curing the polymerizable solution is selected from the group consisting of applying heat to the polymerizable solution, applying radiation to the polymerizable solution, and introducing a catalyst into the polymerizable solution.

19. The method of claim 15, wherein intra-operatively applying the polymerizable solution to the implant comprises pouring the polymerizable solution into a mold, press-fitting the implant into the mold, and closing the mold.

20. The method of claim 19, wherein the polymerizable solution is cured while the implant is held in the closed mold.

21. The method of claim 15, wherein the polymerizable component is a UV-polymerizable component, and curing the polymerizable solution comprises applying UV-radiation to the polymerizable solution.

22. The method of claim 15, wherein providing a sterilized polymerizable component and a sterilized biological component comprises extra-operatively sterilizing the polymerizable component and the biological component.

23. The method of claim 15, wherein the polymerizable solution forms a hydrogel upon curing.

24. The method of claim 23, wherein the hydrogel is selected from polyethylene glycol (PEG) and polyethylene glycol diacrylate (PEGDA).

25. The method of claim 15, wherein the biological component is an osteoinductive or osteoconductive component.

26. The method of claim 15, wherein the polymerizable solution is capable of being cured in less than or about 5 minutes.

27. The method of claim 15, wherein the implant is an orthopedic implant.
